(19) United States
(12) Patent Application Publication
(10) Pub. No.: US 2006/0089317 A1
(43) Pub. Date: Apr. 27, 2006

(54) Treatment of metastatic breast cancer with anthracyclines, and taxanes

(75) Inventor: Lorne J Brandes, Winnipeg, MB (CA)

Correspondence Address: ROTHWELL, FIGG, ERNST & MANBECK, P.C., 1425 K STREET, N.W., SUITE 800, WASHINGTON, DC, 20005, US

(73) Assignee: The University Of Manitoba, Winnipeg, MB (CA)

(21) Appl. No.: 10/526,563

(22) PCT Filed: Sep. 3, 2003
(86) PCT No.: PCT/CA03/01340

§ 371 Date: Aug. 26, 2005

Related U.S. Application Data
(60) Provisional application No. 60/407,679, filed on Sep. 4, 2002.

(51) Int. Cl. A61K 31/704; A61K 31/537; A61K 31/495; A61K 31/337; A61K 31/138

(52) U.S. Cl. 514/34; 514/239.2; 514/252.12; 514/449; 514/651

(57) ABSTRACT

An improved adjuvant treatment of metastatic breast cancer in which patients are administered an anthracycline chemotherapeutic agent and a taxane chemotherapeutic agent is provided in which a diphenyl compound which is a potent antagonist of histamine binding at the intracellular histamine receptor is initially administered prior to administration of the chemotherapeutic agents.

### FIELD OF THE INVENTION

The present invention relates to the treatment of metastatic breast cancer.

### BACKGROUND OF THE INVENTION

One of the major chemotherapeutic treatments is that of malignant growth (cancer) in humans. The objective of chemotherapy is the total extermination of clonogenic tumor or malignant cells, with minimal damage to the patient. However, one of the major limitations of the chemotherapeutic approach for managing human cancer is the general inability of anticancer drugs to discriminate between normal and tumorous cells. Anti-neoplastic agents have the lowest therapeutic indicies of any class of drugs used in humans and hence produce significant and potentially life-threatening toxicities. Certain commonly-used anti-neoplastic agents have unique and acute toxicities for specific tissues. For example, the vinca alkaloids possess significant toxicity for nervous tissues, while adriamycin has specific toxicity for heart tissue and bleomycin has for lung tissue. In general, almost all members of the major categories of anti-neoplastic agents have considerable toxicities for normal cells of gastrointestinal, epidermal and myelopoietic tissues.

Generally, the dose-limiting consideration for chemical management of cancer in humans is the toxicity that anti-neoplastic agents have for the pluripotent stem cells of myelopoietic tissue. This toxicity arises from the fact that most anticancer drugs function preferentially against proliferating cells but with no significant capacity to discriminate between cycling normal and cycling tumor tissues.

In U.S. Pat. Nos. 6,288,799, 5,859,065, 5,708,329, 5,747,543 and 5,618,846, all assigned to University of Manitoba and the disclosures of which are incorporated herein by reference, there is described an improved method for the in vivo chemotherapeutic treatment of cancer in which there is first administered a compound which inhibits normal cell proliferation while promoting maligant cell proliferation, specifically a potent antagonist selective for intracellular histamine receptors, in an amount sufficient to inhibit the binding of intracellular histamine to the receptors in normal and maligant cells. Following sufficient time to permit the inhibition of binding of intracellular histamine, a chemotherapeutic agent is administered. An enhanced toxic effect on the cancer cells from the chemotherapeutic agent is obtained while any adverse effect of the chemotherapeutic agent on normal cells, particularly bone marrow and gastro-intestinal cells, is significantly ameliorated. One useful compound which inhibits normal cell proliferation while promoting malignant cell proliferation is N,N-diethyl-2-[4-(phenylmethyl)-phenoxy]ethanamine, abbreviated herein as DPPE.

### SUMMARY OF INVENTION

It has now surprisingly been found, in a Phase II clinical trial, that patients with metastatic breast cancer treated in accordance with the procedure described in the above-mentioned patents using specific combinations of materials, exhibit enhanced responses to adjuvant chemotherapy.

In the present invention, pretreatment with DPPE and related compounds followed by treatment with a combination of doxorubicin, epirubicin or other anthracyclines and Taxol (paclitaxel), Taxotere (docetaxel) or other taxane, leads to an enhanced anti-cancer effect as compared to the absence of pretreatment with DPPE. In addition, pretreatment with DPPE leads to enhanced survival when compared to the absence of pretreatment with DPPE.

Accordingly, in one aspect, the present invention provides a method of chemotherapy in human patients with metastatic breast cancer, which comprises:

(a) first administering to said patients at least one diphenyl compound of the formula: 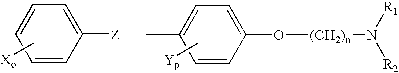
wherein X and Y are each fluorine, chlorine or bromine, Z is an alkylene group of 1 to 3 carbon atoms or ═C═O, or the phenyl groups are joined to form a tricyclic ring, o and p are 0 or 1, R₁ and R₂ are each an alkyl group containing 1 to 3 carbon atoms or are joined together to form a heterocyclic ring with the nitrogen atom and n is 1, 2 or 3, or pharmaceutically-acceptable salts thereof, and

(b) following sufficient time to permit inhibition of binding of intracellular histamine, subsequently administering to the patient an anthracycline chemotherapeutic agent and a taxane therapeutic agent.

In the application of the present invention, the diphenyl compound and the chemotherapeutic agents are generally administered by intravenous infusion. In one preferred procedure, a solution of the diphenyl compound is administered to the patient over a desired period of time prior to administration of the chemotherapeutic agents and a solution of the chemotherapeutic agents in combination with the diphenyl compound then is administered for the period of administration of the chemotherapeutic agents. If desired, a solution of the diphenyl compound is administered after completion of the administration of the chemotherapeutic agents for a desired period of time to ameliorate side effects from the chemotherapeutic agents administration.

### GENERAL DESCRIPTION OF INVENTION

In the present invention, a diphenyl compound is used which is a potent antagonist of histamine binding at the intracellular histamine receptor and is administered in an amount sufficient to inhibit the binding of intracellular histamine at the intracellular binding site (H_{IC}) in normal cells. Such compounds exhibit a pKi of at least about 5, preferably at least about 5.5.

Specific potent compounds which are useful in the present invention are diphenyl compounds of the formula: 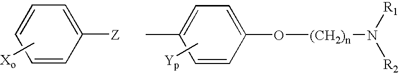
wherein X and Y are each fluorine, chlorine or bromine, Z is an alkylene group of 1 to 3 carbon atoms or ═C═O, o and p are 0 or 1, R₁ and R₂ are each alkyl groups containing 1 to 3 carbon atoms or are joined together to form a hetero-ring with the nitrogen atom and n is 1, 2 or 3. Pharmaceutically-acceptable salts of the diphenyl compounds may be employed.

Alternatively, the benzene rings may be joined to form a tricyclic ring, in accordance with the structure:

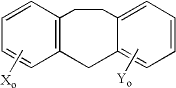

In one preferred embodiment, the group 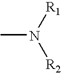
is a diethylamino group, although other alkylamino groups may be employed, such as dimethylamino, and, in another preferred embodiment, a morpholino group, although other heterocyclic ring groups may be employed, such as piperazino. o and p are usually 0 when Z is an alkylene group and n may be 2. In one particularly preferred embodiment, Z is —CH₂—, n is 2, o and p are each 0 and 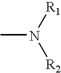
is a diethylamino group. This compound, namely N,N-diethyl-2-[4-(phenylmethyl)-phenoxy]ethanamine, which may be in the form of the free base or in the form of its hydrochloride or other pharmaceutically-acceptable salt, is abbreviated herein as DPPE. In addition to a methylene group linking the benzene rings, other linking groups may be employed, such as ═C═O. Other substitutents may be provided on the benzene rings in addition to the halogen atoms, for example, an imidazole group.

The diphenyl compound employed in the present invention is administered to the patient in any convenient manner, such as by intravenous injection of a solution thereof in an aqueous pharmaceutically-acceptable vehicle. The diphenyl compound is administered to the patient over a period of time before administration of the chemotherapeutic agent.

The chemotherapeutic agents employed herein are a combination of an anthracycline and a taxane. Such anthracyclines are preferably doxorubicin or epirubicin, while the taxanes are preferably Taxol (a trademark of Bristol-Myers Squibb for paclitaxel) or Taxotere (a trademark of Aventis Pharma for docetaxel). The mixture of chemotherapeutic agents is administered in any manner consistent with their normal manner of administration in conventional breast cancer therapy, namely by intravenous infusion of a solution thereof.

The administration of the diphenyl compound to the patient prior to administration of the chemotherapeutic agents is necessary in order to permit the diphenyl compound to inhibit the binding of intracellular histamine in normal and malignant cells and thereby, in effect, shut down the proliferation of the normal cells, but increase proliferation of malignant cells.

The length of time prior to administration of the chemotherapeutic agents that the diphenyl compound is administered depends on the diphenyl compound, its mode of administration and the size of the patient. Generally, the diphenyl compound is administered to the patient for about 30 to about 90 minutes, preferably about 60 minutes, prior to administration of the chemotherapeutic agents.

The quantity of diphenyl compound administered to the patient depends on the side effects to be ameliorated, but should be at least sufficient to inhibit binding of intracellular histamine in normal cells. The quantity required to achieve the beneficial effects of the present invention depends upon the diphenyl compound employed, the chemotherapeutic agent employed and the quantity of such agent employed.

The present invention is able to achieve an enhanced chemotherapeutic effect on cancer cells in a patient with metastatic breast cancer while, at the same time, also protecting normal cells from damage by the chemotherapeutic agents in a wide variety of circumstances where traditional chemotherapy leads to damage of normal cells or tissues not involved in the disease process.

In general, the quantity of diphenyl compound employed in humans is from about 8 to about 320 mg/M² of human to which the diphenyl compound is administered, with about 8 and 240 mg/M² being the optimal dose for gastro-intestinal and bone marrow protection, respectively. Over this dose range, the present invention is able to achieve an enhanced chemotherapeutic effect on breast cancer cells while, at the same time, also protecting normal cells from damage by the chemotherapeutic agents in a wide variety of circumstances where traditional chemotherapy leads to damage of normal cells or tissues not involved in the disease process.

In the treatment of metastatic breast cancer according to the invention, the diphenyl compound preferably is used in an amount of about 3 to about 10 mg/kg of patient administered intravenously over a period of about 30 to about 90 minutes prior to administration of the chemotherapeutic agents and continuing for the period of administration of the chemotherapy agents. In the specific Phase II clinical trial described herein, there was employed 6 mg/kg of DPPE in the form of its hydrochloride salt, administered intravenously as an aqueous solution thereof over 80 minutes, with the last 20 minutes being accompanied by infusion of the chemotherapeutic agents doxorubicin or epirubicin in combination with Taxol or Taxotere followed by the intravenous administration of an aqueous solution at a dose of 2.5 mg/kg of DPPE for 180 minutes accompanied by the infusion of Taxol or for 60 minutes accompanied by the infusion of Taxotere.

A second regimen for DPPE/Taxotere treatment is the intravenous administration of an aqueous solution of DPPE for 80 minutes, with the last 20 minutes being accompanied by infusion of the Taxotere, followed by infusion of Taxotere alone for 40 minutes.

The chemotherapy agents which are employed herein preferably is used in an amount of about 75 to about 225 mg/M² of patient consistent with the identity of the chemotherapy agent. The chemotherapeutic agents may be administered in an amount of about 50 to about 60 mg/M² of patient for doxorubicin or epirubicin, about 175 to about 225 mg/M² of Taxol and about 75 to about 100 mg/M² of Taxotere. In the specific Phase II clinical trial described herein, there was employed 50 mg/M² of doxorubicin/epirubicin and either 175 mg/² Taxol or 75 mg/M² Taxotere administered over the last 20 minutes of infusion of the DPPE solution and over a further 180 minutes for Taxol or 60 minutes for Taxotere, accompanied by infusion of a 2.5 mg/kg of DPPE solution.

Patients with metastatic breast cancer usually are subjected to a number of cycles of chemotherapy at predetermined intervals. The number of cycles for each patient is generally about 5 to about 10 cycles, with about 21 to about 28 days between each cycle.

As set forth herein, a Phase II clinical trial was conducted on patients having metastatic breast cancer in which patients were administered DPPE followed by doxorubicin or epirubicin in combination with Taxol or Taxotere. Various data from the clinical trial were collected and analyzed. Details of the clinical trial are set forth in Example 1 while analysis of the data and comparison to studies not employing DPPE is set forth in Example 2.

### EXAMPLES

### Example 1

This Example describes a Phase II clinical trial involving patients with metastatic breast cancer.

29 patients with metastatic breast cancer were treated with a combination of DPPE and epirubicin/Taxol (N=22), DPPE and doxorubicin/Taxol (N=6) or a combination of DPPE and epirubicin/Taxotere (N=1). DPPE was administered at a dose of 6 mg/Kg over 80 minutes with a combination of epirubicin or doxorubicin administered at a dose of 50 mg/M² and Taxol at a dose of 175 mg/M² or Taxotere at a dose of 75 mg/M² over the last 20 minutes and during a further 180 minutes for Taxol or 60 minutes for Taxotere, at a dose of 2.5 mg/kg. The treatment was repeated at 21 day intervals for 8 to 10 cycles.

The 29 patients with metastatic breast cancer had not been previously treated with taxanes but may have had anthracyclines, or may have had previous adjuvant chemotherapy or tamoxifen. The patients had the demographics shown in Table I. The Tables appear at the end of the descriptive text.

The results obtained are summarized in Tables II and III. As may be seen therein, major responses were observed in 23/29 patients (79%) and some improvement seen in 28/29 patients (97%). The median time to progression (TTP) was 9 months and overall survival (OS) was 26.5 months. These values compare with 5.9 months TTP for a clinical trial utilizing DPPE and doxorubicin (Proc. of ASCO. 2001. Abstract 118) and 23.6 months OS. For the patients receiving DPPE/epirubicin/Taxol (N=22), major responses were observed in 19/22 patients (86%) and overall improvement was observed in 21/22 (96%). The median TTP was 8 months and OS was 30 months.

### Example 2

This Example summarizes the published literature on the use of adjuvant chemotherapy in patients with metastatic breast cancer.

There have been five studies published in which a combination of epirubicin and Taxol or Taxotere have been administered to patients with metastatic breast cancer. These studies are summarized in Table IV. The results contained in these publications can be compared to those set forth in Example 1 and in Tables II and III. Such comparison appears in Table V. In Table V, B₁ is the data summarized in Table II while B₂ is the data summarized in Table III. The data contained in Tables II, III and V may be pooled and such pooling is set forth in Table VI. In Table VI, Brandes refers to the Phase II studies set forth in Example 1 while the other studies refer to those summarized in Table IV.

As may be seen from these data, while the pretreatment with DPPE did not improve the TTP in comparison to patients not receiving DPPE treatment, the overall survival of patients receiving DPPE was significantly improved. In this regard, the median OS in the DPPE study (Example 1) was 33% longer than the pooled median OS in the four studies of epirubicin/Taxotere and epirubicin/Taxol that reported survival data.

As may also be seen from the data presented in Tables IV to VI, the lowest doses of both epirubicin and Taxol were employed in the Phase II study of Example 1. In this regard, the epirubicin dose (50 mg/M²) was 48% of the average dose of (96 mg/M²) used in the five comparator studies.

Despite the lower dosages of epirubicin and Taxol/Taxotere in the study using DPPE (Example 1), the overall response rate was higher than the average overall responses in the published studies that used epirubicin at a dose of 90 mg/M² (79% vs. 65%).

Based on the reported toxicity at the higher doses of epirubicin (75 to 130 mg/M²) employed in the five comparator studies, the toxicity observed in the 29 patients receiving the DPPE/epirubicin/Taxol or Taxotere regimen appeared to be significantly lower.

Despite the small sample size (N=29), the findings of the Phase II study reported in Example I are consistent with those in the clinical trial referred to above, where the addition of DPPE pretreatment appeared to offer a survival advantage without significantly increasing TTP.

### SUMMARY OF INVENTION

In summary of this disclosure, the present invention provides an improved method of treatment of metastatic breast cancer using a combination of anthracyclines and taxanes. Modifications are possible within the scope of the invention.

TABLE I
PHASE II STUDY (1st-LINE)
PATIENT DEMOGRAPHICS

| | |
|---|---|
| Total Patients | 29 |
| Average age: | 50 |
| Disease sites: | Bone 17 |
| | Breast 6 |
| | Chest wall 7 |
| | Liver 8 |
| | Lung/Pleura 7 |
| | Nodes 10 |
| | Ovary 1 |
| | Soft tissue 1 |
| ER Status: | Positive 11 |
| | Negative 15 |
| | Unknown 2 |
| Human epidermal | Positive 6 |
| growth receptor 2: | |

TABLE II
PHASE II STUDY (1st-LINE)
RESULTS

| | | | | | | | TTP | OS |
|---|---|---|---|---|---|---|---|---|
| No. | Cycles | RESPONSE* | | | | | (Mos.) | |
| Patients | (Total) | CR | PR | IMP | ST | PD | Median | |
| 29 | 204 | 5 | 18 | 5 | 0 | 1 | 9** | 26.5*** |

*Major responses = 23/29 (79%); CR/PR/Imp = 28/29 (97%)
**vs. 5.9 mos. median TTP for DPPE/Dox. in MA.19
***vs. 23.6 mos. median OS for DPPE/Dox. in MA.19
TTP (Mean ± SE): 9.1 ± 0.8 mos.
OS (Mean ± SE): 27.1 ± 2.4 mos.

TABLE III
PHASE II 1^{ST}-LINE STUDY RESULTS (CONT.)
DPPE/EPIRUBICIN/TAXOL⁺
(N = 22 ITT/evaluable)

| | | | | | | | TTP | OS |
|---|---|---|---|---|---|---|---|---|
| No. | Cycles | Response* | | | | | (Mos.) | |
| Patients | (Total) | CR | PR | IMP | ST | PD | Median | |
| 22 | 144 | 5 | 14 | 2 | 0 | 1 | 8** | 30*** |

*Major responses = 19/22 (86%); CR/PR/Imp = 21/22 (96%)
**vs. 5.9 mos. median TTP for DPPE/Dox. in MA.19
***vs. 23.6 mos. median OS for DPPE/Dox. in MA.19
⁺Excludes 6 patients who received DPPE/doxorubicin/Taxol and 1 patient who received DPPE/epirubicin/Taxotere

TABLE IV
References

| Author | Journal | Regimen |
|---|---|---|
| 1. Mila-Santos A. et al. | Am J. Clin | N = 31 ITT/Evaluable |
| | Oncol 2001; | Epirubicin 130 mg/m² |
| | 24: 138-142 | Taxotere 100 mg/m² |
| | | q 21 d × 8 cycles max |
| 2. Viens P et al. | Am J. Clin | N = 65 ITT/Evaluable |
| | Oncol 2001; | Epirubicin 60-110 (mean 95) |
| | 24: 328-335 | mg/m² |
| | | Taxotere 75 mg/m² |
| | | q 21 d × 6 cycles max |
| 3. White J. et al. | Clin Oncol | N = 35 ITT/Evaluable |
| | 2000 12: | Epirubicin 75 mg/m² |
| | 256-259 | Taxol 200 mg/m² |
| | | q 21 d × 8 cycles max |
| 4. Grasselli G. et al. | J. Clin Oncol | N = 27 ITT/Evaluable |
| | 2001 | Epirubicin 90 mg/m² |
| | 19: 2222-2231 | Taxol 200 mg/m² |
| | | q 21 d × 9 cycles max |
| 5. Pagani O. et al. | Ann Oncol | N = 70 ITT/Evaluable |
| | 2000 | Epirubicin 90 mg/m² |
| | 11: 985-991 | Taxotere 75 mg/m² |
| | | q 21 d × 8 cycles max |

TABLE V
DPPE/ANTHRACYCLINE/TAXANE
Published Studies

| Ref | N | ORR % | TTP | OS | LINE |
|---|---|---|---|---|---|
| 1 | 32 | 87.5 | 16 | 19.9 | 1 |
| 2 | 62 | 69.4 | 9.1 | 22.7 | 1 |
| 3 | 34 | 50 | 6.4 | 11.4 | 1-2 |
| 4 | 27 | 76 | | 29 | 1 |
| 5 | 68 | 66 | 4.5 | | 1 |
| B₁ | 22 | 86 | 8 | 30 | 1-2 |
| B₂ | 29 | 79 | 9 | 26.5 | 1-2 |

TABLE VI
POOLED STUDY DATA

| | Other | | |
|---|---|---|---|
| | studies | Brandes | |
| Epirubicin (mg/m²) | 96 | 50 | |
| Taxol (mg/m²) | 200 | 175 | |
| Taxotere (mg/m²) | 83.3 | 75 | |
| ORR (%) | 69.8 | 86¹ | 79² |
| Median TTP (mos.) | 9 | 8¹ | 9² |
| Median OS (mos.) | 20.1 | 30¹ | 26.5² |

¹DPPE/Epi/Taxol
²DPPE/epi(doxo)rubicin/Taxol or Taxotere

1. A method of chemotherapy in human patients with metastatic breast cancer, which comprises:
(a) first administering to said patients at least one diphenyl compound of the formula: 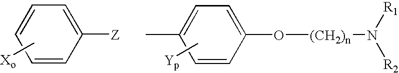
wherein X and Y are each fluorine, chlorine or bromine, Z is an alkylene group of 1 to 3 carbon atoms or ═C═O, or the phenyl groups are joined to form a tricyclic ring, o and p are 0 or 1, R₁ and R₂ are an alkyl each group containing 1 to 3 carbon atoms or are joined together to form a heterocyclic ring with the nitrogen atom and n is 1, 2 or 3, or pharmaceutically-acceptable salts thereof, and
(b) following sufficient time to permit inhibition of binding of intracellular histamine, subsequently administering to the patient an anthracycline chemotherapeutic agent and a taxane chemotherapeutic agent.

2. The method of claim 1 wherein the group 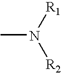
is a diethylamino group, a dimethylamino group, a morpholino group or a piperazino group.

3. The method of claim 1 wherein the group 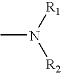
is a diethylamino group, Z is —CH₂, n is 2 and o and p are each O.

4. The method of claim 3 wherein diphenyl compound is in the form of a hydrochloride salt.

5. The method of claim 1 wherein said anthracycline chemotherapeutic agent is doxorubicin or epirubicin.

6. The method of claim 4 wherein said anthracycline chemotherapeutic agent specific for breast cancer is doxorubicin or epirubicin.

7. The method of claim 1, 4, 5 or 6 wherein said taxane chemotherapeutic agent is Taxol or Taxotere.

8. The method of claim 1 wherein said diphenyl compound is administered to the patient about 30 to about 90 minutes prior to said administration of said chemotherapeutic agents.

9. The method of claim 8 wherein said time is about 60 minutes.

10. The method of claim 7 wherein said diphenyl compound is administered by intravenous infusion of a solution thereof over a period of time of up to about 90 minutes prior to administration of said chemotherapeutic agent and is maintained during administration of said chemotherapeutic agent.

11. The method of claim 10 wherein said diphenyl compound is administered for about 60 minutes prior to administration of said chemotherapeutic agent and is maintained during about 20 minutes intravenous infusion of said chemotherapeutic agent.

12. The method of claim 8 wherein said diphenyl compound is administered in an amount of about 0.1 to about 75 mg/kg of said patient.

13. The method of claim 9 wherein said amount is about 3 to about 10 mg/kg of patient.

14. The method of claim 10 wherein said diphenyl compound is administered in an amount of about 3 to about 10 mg/kg of patient.

15. The method of claim 11 wherein said diphenyl compound is administered in an amount of about 6 mg/kg.

16. The method of claim 12 wherein said chemotherapeutic agents are administered in a total amount of about 10 to about 250 mg/M² of patient.

17. The method of claim 15 wherein said anthracycline chemotherapeutic agent is administered in an amount of about 50 mg/M² of patient and taxane chemotherapeutic agent is administered in an amount of 175 mg/M² for Taxol or 75 mg/M² for Taxotere.
